(19) United States
(12) Patent Application Publication
(10) Pub. No.: US 2007/0208259 A1
(43) Pub. Date: Sep. 6, 2007

(54) Patient monitoring alarm escalation system and method

(76) Inventor: Paul D. Mannheimer, Danville, CA (US)

Correspondence Address: Nellcor Puritan Bennett LLC;c/o Fletcher Yoder PC, P.O. BOX 692289, HOUSTON, TX, 77269-2289, US

(21) Appl. No.: 11/369,064

(22) Filed: Mar. 6, 2006

(51) Int. Cl. A61B 5/02

(52) U.S. Cl. 600/500; 600/324

(57) ABSTRACT

Embodiments of the present invention relate to a patient monitoring system and method. Specifically, embodiments of the present invention include a patient monitoring device with a plethysmographic waveform display that includes a Y-axis scale. The Y-axis scale allows that user to qualitatively assess the amplitude of the pulse and thus assess the quality of the pulse signal.

### BACKGROUND OF THE INVENTION

1. Field of the Invention

The present invention relates generally to alarm systems for patient physiological data monitoring instruments. In particular, the present invention relates to a physiological waveform display system for indicating a signal strength or quality based on vertical markers corresponding to waveform amplitudes.

2. Description of the Related Art

This section is intended to introduce the reader to various aspects of art that may be related to various aspects of the present invention, which are described and/or claimed below. This discussion is believed to be helpful in providing the reader with background information to facilitate a better understanding of the various aspects of the present invention. Accordingly, it should be understood that these statements are to be read in this light, and not as admissions of prior art.

In the field of medicine, doctors often desire to monitor certain physiological characteristics of their patients. Accordingly, a wide variety of devices have been developed for monitoring many such characteristics of a patient. Such devices provide doctors and other healthcare personnel with the information they need to provide the best possible healthcare for their patients. As a result, such monitoring devices have become an indispensable part of modern medicine.

One technique for monitoring certain physiological characteristics of a patient is commonly referred to as pulse oximetry, and the devices built based upon pulse oximetry techniques are commonly referred to as pulse oximeters. Pulse oximetry may be used to measure various blood flow characteristics, such as the blood-oxygen saturation of hemoglobin in arterial blood, the volume of individual blood pulsations supplying the tissue, and/or the rate of blood pulsations corresponding to each heartbeat of a patient.

Pulse oximetry typically utilizes a patient monitoring device that, among other functions, displays information related to patient vital signs and provides an audible and/or visual alarm when changes in the vital signs so warrant. This improves patient care by facilitating continuous supervision of a patient without continuous attendance by a human observer (e.g., a nurse or physician).

The accuracy of the estimates of the blood flow characteristics depends on a number of factors. For example, the light absorption characteristics typically vary from patient to patient depending on their physiology. Moreover, the absorption characteristics vary depending on the location (e.g., the foot, finger, ear, and so on) where the sensor is applied, and whether there are objects interfering between the sensor and the tissue location (e.g., hair, nail polish, etc.). Further, the light absorption characteristics vary depending on the design or model of the sensor. Also, the light absorption characteristics of any single sensor design vary from sensor to sensor (e.g., due to different characteristics of the light sources or photo-detector, or both). The clinician applying the sensor correctly or incorrectly may also have a large impact in the results, for example, by loosely or firmly applying the sensor or by applying the sensor to a body part which is inappropriate for the particular sensor design being used.

A marker for the reliability and accuracy of a physiological measurement can be the quality of the signal. Some oximetry devices qualify measurements before displaying them on the monitor, by comparing the measured signals to various phenomenologically-derived criteria. These oximeters qualify the signal by making an assessment of its accuracy and only display values of estimated parameters when the signal quality meets certain criteria. Some commercially available systems display a computed quality index to provide a qualitative or semi-quantitative assessment of signal adequacy. Others interpret the signals and provide messaging text with suggestions to the clinician for improving signal quality. Commonly, the clinician will remove the sensor from a particular tissue location to re-attach it to another location and heuristically repeats this process until more reliable measurements deemed worthy of being displayed are provided by the instrument. While some instruments make estimates of signal quality, there still exists a need for improvements in this area.

### SUMMARY

Certain aspects commensurate in scope with the originally claimed invention are set forth below. It should be understood that these aspects are presented merely to provide the reader with a brief summary of certain forms of the invention might take and that these aspects are not intended to limit the scope of the invention. Indeed, the invention may encompass a variety of aspects that may not be set forth below.

There is provided a monitoring system that includes: a patient monitor adapted to provide a plethysmographic waveform display comprising a Y-axis, wherein the Y-axis comprises a plurality of marks separated by a predetermined spacing.

There is also provided a method that includes: receiving one or more input signals related to a physiological state of a patient; generating a plethysmographic waveform related to the input signals; and displaying the plethysmographic waveform on a display that comprises a Y-axis comprising having a plurality of marks separated by a predetermined spacing.

There is also provided a computer-readable medium that includes computer-executable instructions for performing actions that includes: generating a plethysmographic waveform from patient information; and displaying the plethysmographic waveform on a display comprising a Y-axis having a plurality of marks separated by a predetermined spacing.

### BRIEF DESCRIPTION OF THE DRAWINGS

Advantages of the invention may become apparent upon reading the following detailed description and upon reference to the drawings in which:

FIG. 1 is a perspective view of a patient monitor in accordance with an exemplary embodiment of the present invention;

FIG. 2 is a view of an exemplary waveform display having a Y-axis with tick marks in accordance with an exemplary embodiment of the present invention;

FIG. 3 is a view of an exemplary waveform display of lowered signal quality having a Y-axis with tick marks in accordance with an exemplary embodiment of the present invention;

FIG. 4 is a view of an exemplary waveform having a Y-axis with closely spaced tick marks;

FIG. 5 is a view of an exemplary waveform display having a Y-axis with tick marks that are symmetric about a central mark;

FIG. 6 is a view of an exemplary waveform having a Y-axis with tick marks of unequal lengths;

FIG. 7 is a view of an exemplary waveform having a Y-axis with tick marks and extended dotted lines; and

FIG. 8 is a view of a multiparameter monitor and exemplary patient monitor in accordance with the present techniques.

### DETAILED DESCRIPTION OF SPECIFIC EMBODIMENTS

One or more specific embodiments of the present invention will be described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

The present techniques relate to a patient monitor with plethysmographic (“pleth”) pulse waveform display that includes a Y-axis with tick marks. The tick marks along the Y-axis allow an operator to assess the amplitude of the pulse waveform and thus qualitatively assess the signal strength, quality, or other physical attributes that can be derived or interpreted from the pleth waveform. For example, a clinician may use the pulse waveform signal strength to obtain qualitative insight into trending changes in vasoconstriction or hemodynamics. Further, beat-to-beat variability of the pulse waveform is sometimes used to infer blood volume, which may be related to a variety of clinical states.

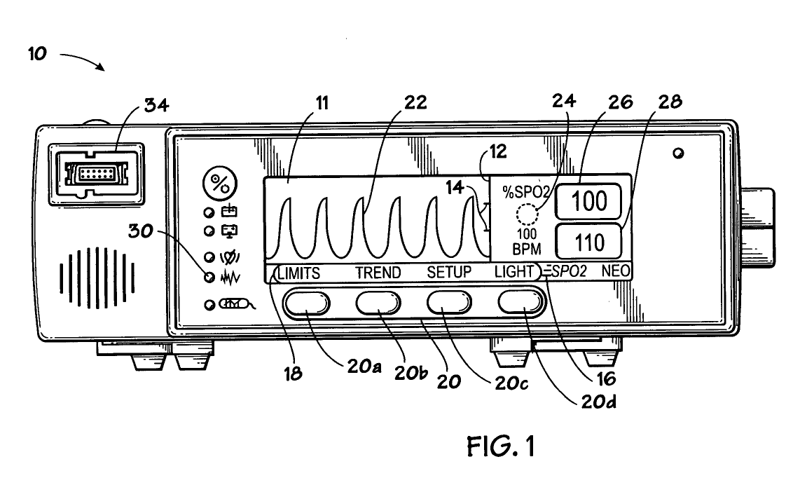

FIG. 1 is a perspective view of a patient monitor 10 in accordance with an exemplary embodiment of the present invention. The monitor includes a display window 11, which may be a cathode ray tube or liquid crystal display, for example. The display window 11 is coupled with the monitor 10 and may display physiological data and other information. For example, a display may include a pleth waveform display 22, an oxygen saturation display 26, and/or a pulse rate display 28 that are displayed in a main display screen or a pleth display screen. The oxygen saturation displays may be a functional arterial hemoglobin oxygen saturation measurement in units of percentage SpO₂. The pulse rate display 28 may indicate a patient's pulse rate in beats per minute. In other embodiments, the display window 11 may show an initial display immediately after the monitor 10 is turned on that includes the general monitor information, such as the serial number of the instrument and the software version.

In general, the monitor 10 includes functions such as processing physiological data or other data received from a patient sensor (discussed below) via a cable connection port 34 that is configured to communicatively couple with the sensor. The monitor 10 may be processor-based and software-controlled. The software may be stored in memory, such as RAM, ROM, flash, or on ASIC. Additionally, the monitor 10 may be re-programmed. The physiological data may be processed and the output displayed in the display window 11. In addition to displaying physiological information, the monitor 10 may also display information related to alarms and monitor settings. For example, in some embodiments, the monitor 10 employs SatSeconds™ by Nellcor™ to detect alarms and manage nuisance alarms. SatSeconds™ may include activation of an alarm based on limits that may include the integral of time and depth of a desaturation event and may also include an indicator 24 that may serve to inform the operator that an SpO₂ reading has been detected outside of the limit settings. The monitor may also include other settings relating to signal quality, such as a signal quality indicator light 30. The display may also include an alarm status indicator (not shown), and special settings such as a fast response mode setting indicator 16.

The monitor 10 may include a number of keys that are related to the operating functions. The keys may include fixed function key sand programmable function keys (“soft keys”) 20, and associated soft key icons in the soft key menu 18. The four soft keys 20a, 20b, 20c, and 20d are pressed to select a corresponding one of the soft key icons. The soft key icon menu 18 indicates which software menu items can be selected through the soft keys 20. Pressing a soft key 20 associated with, such as below, above, or next to an icon, selects the option.

In certain embodiments, the monitor 10 may include computer-executable instructions for allowing an operator to specify the tick mark separation. Such instructions may include steps for determining how many tick marks will be used, depending on the length of the Y-axis. The user may select the soft key 20c associated with the SETUP soft key icon in the soft key menu bar 18 to access a settings menu that may contain further user-input options for either selecting a predetermined value, or, alternatively, removing the Y-axis from the display.

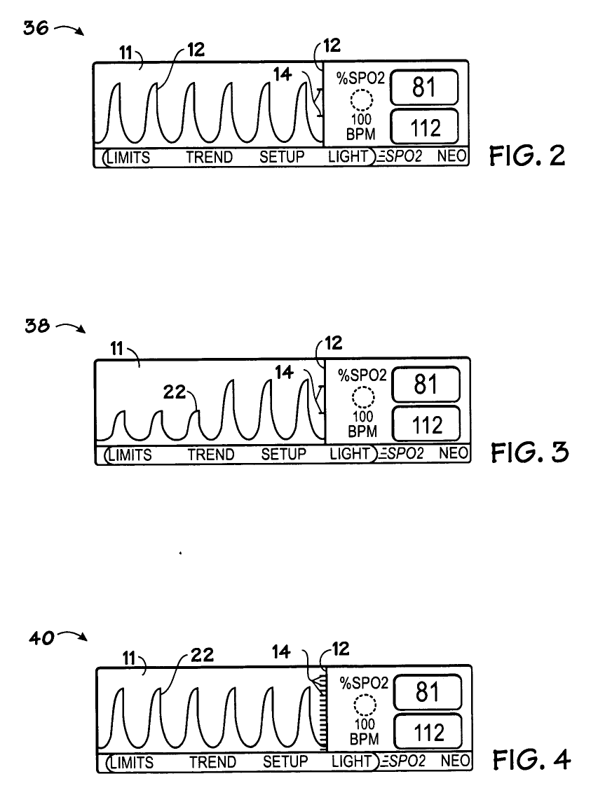

FIG. 2 is a view of an exemplary pleth waveform display 38 with relatively constant amplitude. The Y-axis 12 includes tick marks 14 that may be disposed along the Y-axis at a predetermined value. The predetermined value and the scale of the Y-axis may correspond with a previously determined scale that corresponds with good signal quality. This scale may be clinically or empirically determined and stored in the monitor's processor, or may be calibrated for an individual patient. In certain embodiments the tick marks on the Y-axis may correspond to a quantitative scale of measure such as pulse amplitude units. A pulse amplitude unit may be the rounded integer value of 10 * IR percent modulation, where the percent modulation is calculated from the AC/DC sensor signal output. For example, the tick marks may be dispersed along the Y-axis every 1-10 pulse amplitude units. Alternatively, the Y-axis scale may correspond directly to the modulation amplitude in the units of measure of percent modulation.

FIG. 3 is an example of an exemplary pleth waveform display 38 with variable signal strength and quality. The pleth waveform is shown to vary in amplitude over time. Such variation in waveform amplitude may be associated with a change in clinical state, change in sensor positioning, or other factor that may affect the signal. An operator may assess the change in amplitude by observing the relative change in the amplitude as compared to the tick marks 14. For example, a clinician may wish to monitor ventilator-induced or therapeutic agent-induced changes in pulse amplitude, or simply verify the sensor is properly placed on the patient.

FIG. 4 shows an exemplary pleth waveform display 40 with a Y-axis 42 having tick marks 14 that are disposed along the Y-axis 12 at a predetermined spacing. As shown the tick marks 14 relatively close together. Such a configuration may be useful for determining relatively small changes in pulse amplitude that may be associated with, for example, signal artifacts.

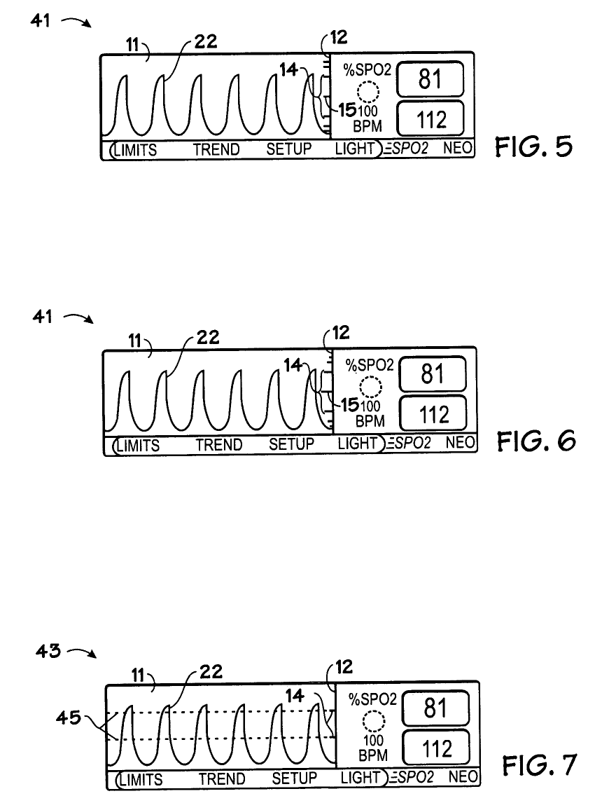

FIG. 5 shows an exemplary pleth waveform display 41 with a Y-axis having tick marks 14 that are disposed along the Y-axis 12 with a non-linear spacing. Such a non-linear spacing may be a log or other non-linear scale. For example, the spacing may be nonlinear in order to correlate to nonlinear scaling of the pleth waveform 22. In certain embodiments, the pleth waveform 22 may be scaled piecewise such that certain ranges of amplitude of the waveform 22 are scaled with one scale, while other amplitudes ranges are displayed on a different scale. In such an embodiment, the piecewise pleth waveform 22 is displayed using a Y-axis 12 that may need to account for the presence of multiple scales along different portions of the waveform 22. This may be useful for emphasizing portions of the waveform 22 that are likely to provide more information for the operator and providing more tick marks 14 in those portions. As shown, the tick marks 14 are symmetric about a center mark 15. The center mark 15 may indicate the mid-point of the Y-axis.

As shown in FIG. 6, the length of each of the tick marks 14 may vary along the Y-axis 12. By utilizing differing tick mark lengths, the center of the scale may be better distinguished, or major and minor scaling units can be delineated. As above, the tick marks 14 may be symmetric about a center mark 15.

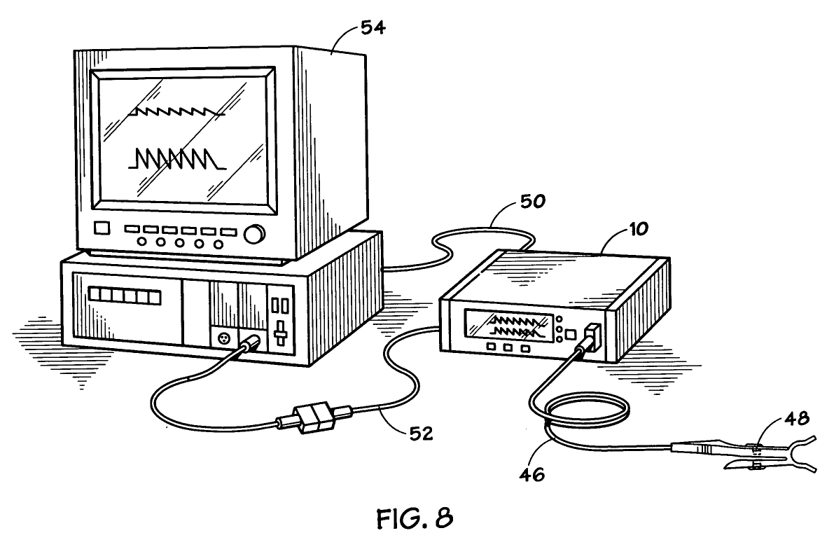

FIG. 7 shows an alternate embodiment of a display 43 in which the Y-axis includes a plurality of phantom or dotted lines 45 that are separated by a predetermined spacing. These lines may provide a reference point across several peaks of the pleth waveform 22. Such an embodiment may be useful in providing a pleth waveform display in which changes in amplitude are easily discerned at a glance.

The exemplary pulse oximetry monitor 10 described herein may be used with a sensor 48, as illustrated in FIG. 8. It should be appreciated that the cable 46 of the sensor 48 may be coupled to the monitor 10 or it may be coupled to a transmission device (not shown) to facilitate wireless transmission between the sensor 48 and the monitor 10. The sensor 48 may be any suitable sensor 48, such as those available from Nellcor Puritan Bennett Inc. Furthermore, to upgrade conventional pulse oximetry provided by the monitor 10 to provide additional functions, the monitor 10 may be coupled to a multi-parameter patient monitor 54 via a cable 50 connected to a sensor input port or via a cable 52 connected to a digital communication port. The monitor 10 may also be connected to a printer (not shown) that may print a monitor display as described herein. For example, the printed monitor display may include a pleth waveform that includes a Y-axis with tick marks.

While the invention may be susceptible to various modifications and alternative forms, specific embodiments have been shown by way of example in the drawings and will be described in detail herein. However, it should be understood that the invention is not intended to be limited to the particular forms disclosed. Rather, the invention is to cover all modifications, equivalents and alternatives falling within the spirit and scope of the invention as defined by the following appended claims.

What is claimed is:

1. A monitoring system, comprising:
a patient monitor adapted to provide a plethysmographic waveform display comprising a Y-axis, wherein the Y-axis comprises a plurality of marks separated by a predetermined spacing comprising a quantitative scale of measure.

2. The monitoring system of claim 1, wherein the predetermined spacing is linearly related to pulse amplitude.

3. The monitoring system of claim 1, wherein the predetermined spacing is non-linearly related to pulse amplitude.

4. The monitoring system of claim 1, wherein the predetermined spacing comprises a logarithmic relationship to the pulse amplitude.

5. The monitoring system of claim 1, wherein the predetermined spacing is adapted to be selected by an operator.

6. The monitoring system of claim 1, wherein the marks comprise a tick marks.

7. The monitoring system of claim 6, wherein the tick marks are symmetric about a center mark on the Y-axis.

8. The monitoring system of claim 6, wherein the tick marks comprise a plurality of lengths.

9. The monitoring system of claim 1, wherein the marks comprise lines.

10. The monitoring system of claim 1, wherein the patient monitor comprises a pulse oximetry monitor.

11. A method comprising:
receiving one or more input signals related to a physiological state of a patient;
generating a plethysmographic waveform related to the input signals; and
displaying the plethysmographic waveform on a display that comprises a Y-axis having a plurality of marks separated by a predetermined spacing, the predetermined spacing comprising a quantitative scale of measure.

12. The method of claim 11, wherein the predetermined spacing is linearly related to the pulse amplitude.

13. The method of claim 11, wherein the predetermined spacing is non-linearly related to the pulse amplitude.

14. The method of claim 11, wherein the marks comprises tick marks.

15. The method of claim 11, wherein the marks comprise lines.

16. A computer-readable medium comprising computer-executable instructions for performing actions comprising:
generating a plethysmographic waveform from patient information; and
displaying the plethysmographic waveform on a display comprising a Y-axis having a plurality of marks separated by a predetermined spacing comprising a quantitative scale of measure.

17. The computer readable medium of claim 16, comprising computer-executable instructions for changing the predetermined spacing.

18. The computer readable medium of claim 16, comprising computer-executable instructions for selecting the predetermined spacing from a user-input.
